(19) United States
(12) Patent Application Publication
(10) Pub. No.: US 2025/0352396 A1
(43) Pub. Date: Nov. 20, 2025

(54) IMPROVED DRESSINGS, SYSTEMS, AND APPARATUS FOR SENSING PRESSURE AT A TISSUE SURFACE

(71) Applicant: Solventum Intellectual Properties Company, Maplewood, MN (US)

(72) Inventors: Benjamin A. PRATT, Poole (GB); Christopher BREACH, Bracknell (GB)

(21) Appl. No.: 18/872,106

(22) PCT Filed: May 31, 2023
(86) PCT No.: PCT/IB2023/055601

§ 371 Date: Dec. 5, 2024

Related U.S. Application Data
(60) Provisional application No. 63/349,273, filed on Jun. 6, 2022.

(51) Int. Cl. A61F 13/0203; A61F 13/00

(52) U.S. Cl. CPC A61F 13/0226; A61F 13/00051; A61F 2013/00089

(57) ABSTRACT

A dressing configured to be positioned adjacent to a tissue site can include a cover, a tissue interface, a dressing interface, and a sensing conduit. The dressing interface can be configured to be coupled to the cover and can include a housing, a fluid pathway, and a sensing pathway. The fluid pathway can extend internally through the housing between a fluid inlet cavity and a fluid outlet port. The sensing pathway can extend internally through the housing between a sensing inlet port and a sensing outlet port with the sensing pathway being fluidly isolated from the fluid pathway. The sensing conduit can be configured to be coupled between the sensing inlet port and a tissue contact surface of the tissue interface. Also disclosed are systems, apparatuses, and methods suitable for use with various example dressings.

### CROSS-REFERENCE TO RELATED APPLICATIONS

This application is a U.S. National Stage Entry of PCT International Application No. PCT/IB2023/055601, filed on May 31, 2023, which claims the benefit of priority to U.S. Provisional Application No. 63/349,273, filed on Jun. 6, 2022, which is incorporated herein by reference in its entirety.

### TECHNICAL FIELD

The invention set forth in the appended claims relates generally to tissue treatment systems and more particularly, but without limitation, to dressings, systems, and apparatus for treating tissue with negative-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful dressings, systems, apparatuses, and methods for sensing pressure at a tissue interface or surface treated with negative-pressure therapy are set forth in the appended claims. Illustrative example embodiments are provided to enable a person skilled in the art to make and use the claimed subject matter.

In some examples, a dressing configured to be positioned adjacent to a tissue site can include a cover, a tissue interface, a dressing interface, and a sensing conduit. The cover can be configured to create a seal at the tissue site. The tissue interface can include a tissue contact surface configured to be positioned in contact with the tissue site. The dressing interface can be configured to be coupled to the cover. The dressing interface can include a housing, a fluid pathway, and a sensing pathway. The housing can include a mounting surface surrounding a fluid inlet cavity and a sensing inlet port. The fluid pathway can extend internally through the housing between the fluid inlet cavity and a fluid outlet port external to the housing. The sensing pathway can extend internally through the housing between the sensing inlet port and a sensing outlet port external to the housing with the sensing pathway being fluidly isolated from the fluid pathway. The sensing conduit can be configured to be coupled between the sensing inlet port and the tissue contact surface of the tissue interface.

In some examples, a system can include a dressing, a negative-pressure source, and a pressure sensor. The dressing can be configured to be positioned adjacent to a tissue site and can include a cover, a tissue interface, a dressing interface, and a sensing conduit. The cover can be configured to create a seal at the tissue site. The tissue interface can include a tissue contact surface configured to be positioned in contact with the tissue site. The dressing interface can be configured to be coupled to the cover. The dressing interface can include a housing, a fluid pathway, and a sensing pathway. The housing can include a mounting surface surrounding a fluid inlet cavity and a sensing inlet port. The fluid pathway can extend internally through the housing between the fluid inlet cavity and a fluid outlet port external to the housing. The sensing pathway can extend internally through the housing between the sensing inlet port and a sensing outlet port external to the housing with the sensing pathway being fluidly isolated from the fluid pathway. The sensing conduit can be configured to be coupled between the sensing inlet port and the tissue contact surface of the tissue interface. The negative-pressure source can be configured to be fluidly coupled to the fluid pathway, and the pressure sensor can be configured to be fluidly coupled to the sensing pathway.

In some examples, a dressing interface can be configured to be coupled to a dressing. The dressing interface can include a housing and a sensing conduit. The housing can be configured to be coupled to a first portion of the dressing. The housing can include a fluid pathway and a sensing pathway. The sensing conduit can be configured to pass into a thickness of the dressing. The sensing conduit can include a first end configured to be coupled to the sensing pathway and a second end including a flange that is configured to be coupled to a second portion of the dressing. The sensing pathway can extend through the sensing conduit to the second portion of the dressing with the sensing pathway being fluidly isolated from the fluid pathway.

Objectives, advantages, and a preferred mode of making and using the claimed subject matter may be understood best by reference to the accompanying drawings in conjunction with the following detailed description of illustrative example embodiments.

### BRIEF DESCRIPTION OF THE DRAWINGS

FIG. 1 is a block diagram of an example embodiment of a therapy system that can provide negative-pressure treatment and optional instillation treatment in accordance with this specification;

FIG. 2 is an exploded view of an example of the dressing of FIG. 1, illustrating additional details that may be associated with some example embodiments;

FIG. 3 is an isometric view of the dressing of FIG. 2, as assembled;

FIG. 4 is an isometric view of the dressing of FIG. 2, as assembled, and with an example dressing interface attached;

FIG. 5 is a top view of an assembled example dressing according to this specification;

FIG. 6 is a bottom view of the assembled example dressing of FIG. 5;

FIG. 7 is a top view illustrating additional details that may be associated with some examples of a tissue interface according to this specification;

FIG. 8A is an isometric view of an example dressing interface and sensing conduit according this specification;

FIG. 8B is an isometric, cross-sectional view of the dressing interface and sensing conduit of FIG. 8A, illustrating a cross-section of an example fluid pathway;

FIG. 8C is an isometric, cross-sectional view of the dressing interface and sensing conduit of FIG. 8A, illustrating a cross-section of an example sensing pathway;

FIG. 9A is a cross-sectional view of the example dressing of FIG. 4, taken at line 9A-9A, applied to an example tissue site, and illustrating additional details associated with some examples of the therapy system of FIG. 1;

FIG. 9B is a detail view, taken at reference 9B in FIG. 9A, illustrating additional features, which may be associated with some examples of the dressing of FIG. 9A;

FIG. 9C is a detail view illustrating additional features, which may be associated with the detail view of FIG. 9B in some implementations of the dressing of FIG. 9A;

FIG. 10 is a detail view of an example dressing, dressing interface, and sensing conduit positioned at a tissue site according to this specification, illustrating additional details that can be associated with some embodiments;

FIG. 11 is a detail view of another example dressing, dressing interface, and sensing conduit positioned at a tissue site according to this specification, illustrating additional details that can be associated with some embodiments; and

FIG. 12 is a detail view of the example dressing, dressing interface, and sensing conduit of FIG. 11, illustrating additional details that can be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

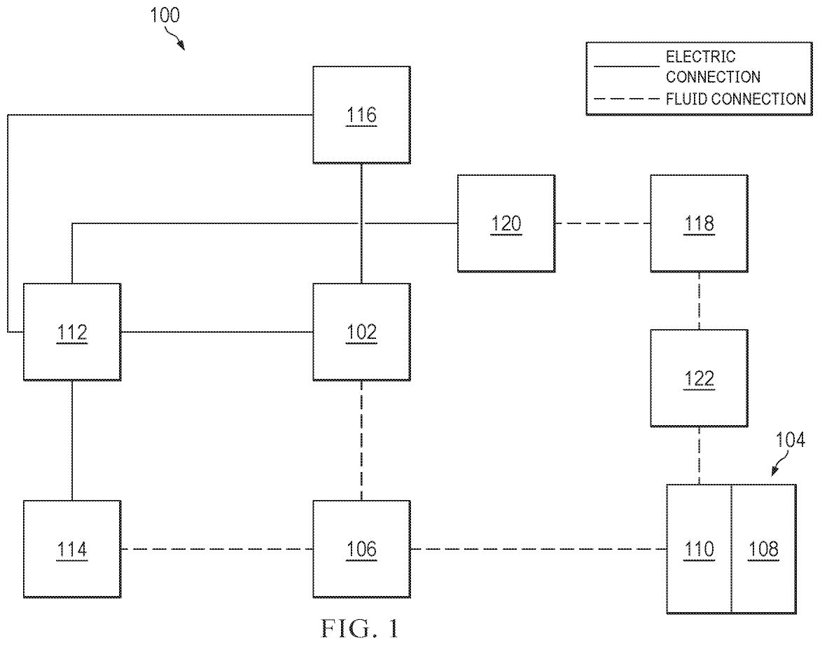

FIG. 1 is a block diagram of an example embodiment of a therapy system 100 that can provide negative-pressure therapy optional instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104, or a portion of the dressing 104, such as the cover 110.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

The therapy system 100 may also optionally include a source of instillation solution. For example, a solution source 118 may be fluidly coupled to the dressing 104, as illustrated in the example embodiment of FIG. 1. The solution source 118 may be fluidly coupled to a positive-pressure source such as a positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 118, and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively or additionally, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between-50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, may be any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, for example, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

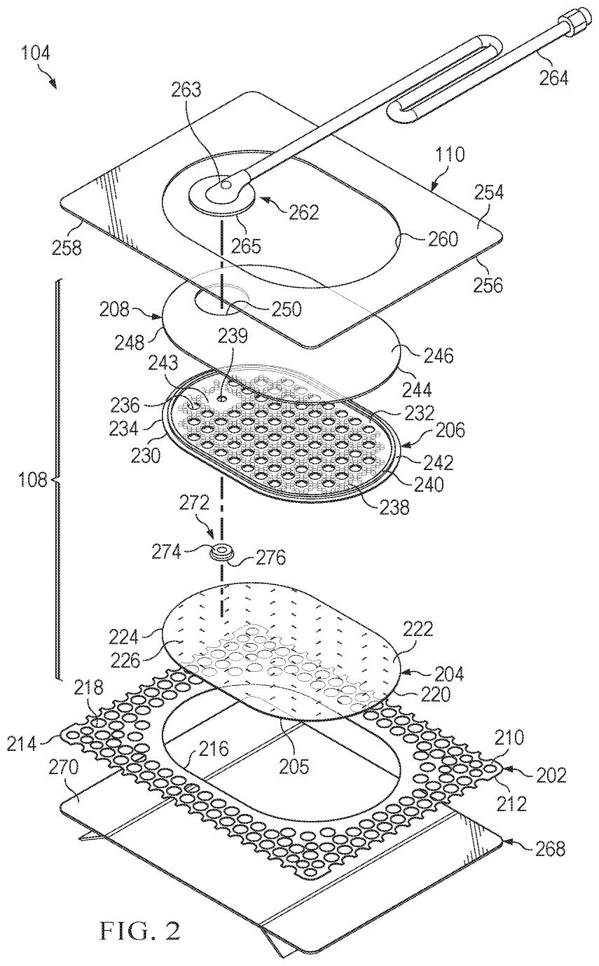

The tissue interface 108 may include a tissue contact surface 205, shown in FIG. 2, that can be generally adapted to partially or fully contact a tissue site. The tissue interface 108 may take many forms, may include multiple layers of material and features, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid through the tissue interface 108 under pressure. For example, a manifold may be adapted to receive negative pressure from a source and distribute negative pressure through multiple apertures through the tissue interface 108, which may have the effect of collecting fluid from a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface 108 may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inspire 2327 polyurethane films, commercially available from Transcontinental Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable include hypochlorite-based solutions, silver nitrate (0.5%), sulfur-based solutions, biguanides, cationic solutions, and isotonic solutions.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The process of reducing pressure may be described illustratively herein as “delivering,” “distributing,” or “generating” negative pressure, for example. In general, exudate and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure.

Negative pressure applied to the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, the controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

In some embodiments, the controller 112 may have a continuous pressure mode, in which the negative-pressure source 102 is operated to provide a constant target negative pressure for the duration of treatment or until manually deactivated. Additionally or alternatively, the controller may have an intermittent pressure mode. In example embodiments, the controller 112 can operate the negative-pressure source 102 to cycle between a target pressure and atmospheric pressure. For example, the target pressure may be set at a value of 135 mmHg for a specified period of time (e.g., five minutes), followed by a specified period of time (e.g., two minutes) of deactivation. The cycle can be repeated by activating the negative-pressure source 102, which can form a square wave pattern between the target pressure and atmospheric pressure.

In some example embodiments, the increase in negative-pressure from ambient pressure to the target pressure may not be instantaneous. For example, the negative-pressure source 102 and the dressing 104 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise rate of negative pressure set at a rate of 25 mmHg/min. and a descent rate set at 25 mmHg/min. In other embodiments of the therapy system 100, the triangular waveform may vary between negative pressure of 25 and 135 mmHg with a rise rate of about 30 mmHg/min. and a descent rate set at about 30 mmHg/min.

In some embodiments, the controller 112 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 112, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 112 may receive and process data, such as data related to instillation solution provided to the tissue interface 108. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 112 may also control the operation of one or more components of the therapy system 100 to instill solution. For example, the controller 112 may manage fluid distributed from the solution source 118 to the tissue interface 108. In some embodiments, fluid may be instilled to a tissue site by applying a negative pressure from the negative-pressure source 102 to reduce the pressure at the tissue site, drawing solution into the tissue interface 108. In some embodiments, solution may be instilled to a tissue site by applying a positive pressure from the positive-pressure source 120 to move solution from the solution source 118 to the tissue interface 108. Additionally or alternatively, the solution source 118 may be elevated to a height sufficient to allow gravity to move solution into the tissue interface 108.

The controller 112 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 108, or it may be implemented to provide a dynamic pressure mode of operation to vary the flow rate of instillation solution through the tissue interface 108. Alternatively, the application of negative pressure may be implemented to provide an intermittent mode of operation to allow instillation solution to dwell at the tissue interface 108. In an intermittent mode, a specific fill volume and dwell time may be provided depending, for example, on the type of tissue site being treated and the type of dressing being utilized. After or during instillation of solution, negative-pressure treatment may be applied. The controller 112 may be utilized to select a mode of operation and the duration of the negative pressure treatment before commencing another instillation cycle by instilling more solution.

FIG. 2 is an exploded view of an example of the dressing 104 of FIG. 1, illustrating additional details that may be associated with some embodiments. In the example of FIG. 2, the dressing 104 may include a sealing layer 202, a first film layer 204, a manifold layer 206, a second film layer 208, and the cover 110. In some examples, the first film layer 204, the manifold layer 206, and the second film layer 208 may form the tissue interface 108 of the dressing 104. Further, the second film layer 208 may additionally or alternatively form a portion of the cover 110. The sealing layer 202 may be formed from a soft, pliable material suitable for providing a fluid seal with a tissue site, such as a suitable gel material, and may have a substantially flat surface. In various implementations, the sealing layer 202 may include, without limitation, a silicone gel, a soft silicone, hydrocolloid, hydrogel, polyurethane gel, polyolefin gel, hydrogenated styrenic copolymer gel, a foamed gel, soft closed-cell foams such as polyurethanes and polyolefins coated with adhesives, polyurethane, polyolefin, or hydrogenated styrenic copolymers. In various implementations, the sealing layer 202 may have a thickness in a range of about 200 micrometers to about 1,000 micrometers. In various implementations, the sealing layer 202 may be formed from hydrophobic or hydrophilic materials.

In various implementations, the sealing layer 202 may include or be formed from a hydrophobic or hydrophobic-coated material. For example, the sealing layer 202 may be formed by coating a spaced material, such as woven, nonwoven, molded, or extruded mesh, with a hydrophobic material such as a soft silicone.

The sealing layer 202 may have a top surface 210 opposite a bottom surface 212, a periphery 214 defined by an outer perimeter of the sealing layer 202, and a treatment aperture 216 formed through the sealing layer 202. In various implementations, the treatment aperture 216 may have an outline complementary to or corresponding to an outer perimeter of the manifold 206. The scaling layer 202 may also include a plurality of apertures 218 formed through the sealing layer 202. In various implementations, the plurality of apertures 218 may be formed through a region of the sealing layer 202 between the treatment aperture 216 and the periphery 214.

In various implementations, the apertures 218 may be formed by cutting, perforating, or applying local radio-frequency or ultrasonic energy through the sealing layer 202. In various implementations, the apertures 218 may be formed by other suitable techniques for forming an opening in the sealing layer 202. In various implementations, the apertures 218 may have a uniform distribution pattern, or may be randomly distributed. In various implementations, the apertures 218 may have many any combination of shapes, including circles, squares, stars, ovals, polygons, slits, complex curves, rectilinear shapes, or triangles.

In various implementations, each of the apertures 218 may have uniform or similar geometric properties. For example, each of the apertures 218 may be a circular aperture, and have substantially the same diameter. In various implementations, each of the apertures 218 may have a diameter in a range of between about 1 millimeter and about 20 millimeters.

In various implementations, the geometric properties of the apertures 218 may vary. For example, the diameters of the apertures 218 may vary depending on the positioning of the respective apertures 218 in the sealing layer 202. In various implementations, at least some of the apertures 218 may have a diameter in a range of between about 5 millimeters to about 10 millimeters. In various implementations, at least some of the apertures 218 may have a diameter in a range of between about 7 millimeters and about 9 millimeters. In various implementations, the sealing layer 202 may include corners, and the apertures 218 disposed at or near the corners may have diameters in a range of between about 7 millimeters and about 8 millimeters.

In various implementations, at least some of the apertures 218 positioned near the periphery 214 may have an interior that is cut open or exposed at the periphery 214 and is in lateral communication in a lateral direction (relative to the top surface 210 and/or bottom surface 212) with the periphery 214. In various implementations, the lateral direction may refer to a direction in a same plane as the top surface 210 and/or bottom surface 212 and extending towards the periphery 214. In various implementations, at least some of the apertures 218 positioned proximate to or at the periphery 214 may be spaced substantially equidistantly around the periphery 214. Alternatively, in various implementations, the spacing of the apertures 218 proximate to or at the periphery 214 may be spaced irregularly.

The first film layer 204 may include a suitable structure for controlling or managing fluid flow. In various implementations, the first film layer 204 may be a fluid-control layer that includes a liquid-impermeable, vapor-permeable elastomeric material. In various implementations, the first film layer 204 may be formed from or include a polymer film. For example, in various implementations, the first film layer 204 may be formed from or include a polyolefin film, such as a polyethylene film. In various implementations, the first film layer 204 may be substantially clear or optically transparent. In various implementations, the first film layer 204 may be formed from or include the same material as the cover 110. In various implementations, the first film layer 204 may be formed from or include a biocompatible polyurethane film tested and certified according to the USP Class VI Standard. In various implementations, the first film layer 204 may also have a smooth or matte surface texture. In various implementations, the first film layer 204 may have a glossy or shiny finish equal to or exceeding a grade B3 according to the Society of Plastics Industry (SPI) standards. In various implementations, the surface of the first film layer 204 may be a substantially flat surface, with height variations in a range of about 0.2 millimeters to about 1 centimeter.

In various implementations, the first film layer 204 may be hydrophobic. The hydrophobicity of the first film layer 204 may vary, but may have a contact angle with water of at least 90 degrees in some examples. In various implementations, the first film layer 204 may have a contact angle with water of no more than 150 degrees. In various implementations, the first film layer 204 may have a contact angle with water in a range of about 90 degrees to about 120 degrees, or in a range of about 120 degrees to about 150 degrees. Water contact angle may be measured using any standard apparatus. Although manual goniometers may be used to visually approximate contact angles, contact angle measuring instruments may often involve integrated systems that include a level stage, a liquid dropper (such as a syringe), a camera, and software designed to calculated contact angles more accurately and precisely. Non-limiting examples of such integrated systems include the FTÅ125, FTÅ200, FTÅ2000, and FTÅ4000 systems, all commercially available from First Ten Angstroms, Inc., of Portsmouth, Virginia, and the DTA25, DTA30, and DTA100 systems, all commercially available from Kruss GmbH of Hamburg, Germany. Unless otherwise specified, water contact angles herein are measured using deionized and/or distilled water on a level sample surface for a sessile drop added from a height of no more than five centimeters in air at 20-25° C. and 20-50% relative humidity. Contact angles herein represent averages of five to nine measured values, with the highest and lowest measure values discarded. In various implementations, the hydrophobicity of the first film layer 204 may be further enhanced with a hydrophobic coating of other materials, such as silicones and fluorocarbons.

The first film layer 204 may also be suitable for welding to other layers, including the manifold layer 206 and the second film layer 208. In various implementations, the first film layer 204 may be adapted for welding to polymers such as polyurethane, polyurethane films, and polyurethane foams using heat welding, radio-frequency (RF) welding, ultrasonic welding, or other methods. RF welding may be particularly suitable for more polar materials, such as polyurethane, polyamides, polyesters, and acrylates. Sacrificial polar interfaces may be used to facilitate RF welding of less polar film materials, such as polyethylene.

The area density of the first film layer 204 may vary according to a prescribed therapy or application. In various implementations, an area density of less than 40 grams per square meter may be suitable. In various implementations, the area density of the first film layer 204 may be in a range of about 20 grams per square meter to about 30 grams per square meter.

In various implementations, the first film layer 204 may be formed from or include a hydrophobic polymer, such as a polyethylene film. The simple and inert structure of polyethylene provides a surface that interacts little, if any, with biological tissues and fluids, and provides a surface that may encourage the free flow of liquids and exhibits a low adherence to tissues and fluids, properties that may be particularly advantageous for many applications. In various implementations, the first film layer 204 may be formed from other polymeric films such as polyurethanes, acrylics, polyolefins (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate styreneics, silicones, fluoropolymers, and acetates. In various implementations, the first film layer 204 may have a thickness in a range of about 20 micrometers to about 500 micrometers. In various implementations, the first film layer 204 may have a thickness of about 23 micrometers, about 25 micrometers, about 100 micrometers, about 250 micrometers, about 300 micrometers, and about 500 micrometers. In various implementations, the first film layer 204 may include a polar film suitable for lamination to the polyethylene film, such as polyamides, co-polyesters, ionomers, and acrylics. In various implementations, the first film layer 204 may include a tie layer to improve the bond between the polyethylene and polar film layers. In various implementations, the tie layer may include ethylene vinyl acetate or modified polyurethanes. In various implementations, the first film layer 204 may include an ethyl methyl acrylate (EMA) film.

As illustrated in FIG. 2, the first film layer 204 may have a first side or bottom surface 220 opposite a second side or top surface 222, and a periphery 224 defined by an outer perimeter of the first film layer 204. In some examples, the bottom surface 220 of the first film layer 204 may define the tissue contact surface 205 of the tissue interface 108. In various implementations, the periphery 224 may be a stadium, discorectangular, or obround shape. The first film layer 204 may also include one or more fluid passages 226 formed or disposed through the bottom surface 220 and the top surface 222 of first film layer 204, and which may be distributed uniformly or randomly across the first film layer 204.

In various implementations, the fluid passages 226 may function as bi-directional and fluid-responsive valves. For example, each fluid passages 226 may be an clastic passage that is normally unstrained to prevent or substantially reduce fluid flow across the fluid passage 226, and can expand or open to allow fluid flow across the fluid passage 226 in response to a pressure gradient applied across the fluid passage 226. In various implementations, the fluid passages 226 may include perforations formed in the first film layer 204. Perforations may be formed by removing material from the first film layer 204, or cutting through the first film layer 204. In various implementations, cutting through the first film layer 204 may deform the edges of the perforations. In various implementations, the fluid passages 226 may be sufficiently narrow to form a seal or a fluid restriction to substantially reduce or prevent fluid flow across the fluid passage 226, particularly in the absence of a pressure differential. In various implementations, one or more of the fluid passages 226 may be an elastomeric valve that is normally closed when unstrained to prevent liquid flow across the valve, and that can open in response to a pressure gradient. In various implementations, the fluid passages 226 may include fenestrations formed through the first film layer 204. Fenestrations may be formed by removing material from the first film layer 204, but the amount of material removed and the resulting dimensions of the fenestrations may be up to an order of magnitude less than perforations, and may not deform the edges.

In various implementations, the fluid passages 226 may include one or more slits, slots, or combinations of slits and slots in the first film layer 204. In various implementations, the fluid passages 226 may include linear slots having a length less than about five millimeters and a width less than about two millimeters. In various implementations, the length may be at least about two millimeters, and the width may be at least about 0.5 millimeters. In various implementations, the length may be in a range of about two millimeters to about five millimeters and the width may be in a range of about 0.5 millimeters to about two millimeters, with a tolerance of about 0.1 millimeters. In various implementations, the length may be about three millimeters. Such dimensions and tolerances may be achieved with a laser cutter, for example. In various implementations, slots of such configurations may function as imperfect valves that substantially reduce liquid flow in a normally closed or resting state. Such slots may form a flow restriction without being completely closed or sealed. The slots can expand or open wider in response to a pressure gradient applied across the slot to allow increased liquid flow through the slot.

In various implementations, the fluid passages 226 may include linear slits having a length of less than about five millimeters. In various implementations, the length of the linear slits may be at least about two millimeters. In various implementations, the length of the linear slits may be in a range of about two millimeters to about five millimeters, with a tolerance of about 0.1 millimeters. In various implementations, the length of the linear slits may be about three millimeters.

In various implementations, the manifold layer 206 may be formed as a substantially sheet-like structure having a first side or bottom surface 230 opposite a second side or top surface 232, and a periphery 234 defined by an outer perimeter of the manifold layer 206. In various implementations, the periphery 234 of the manifold layer 206 may be substantially similar to or coextensive with the periphery 224 of the first film layer 204. Further, the bottom surface 230 of the manifold 206 may be disposed adjacent to the top surface 222 of the first film layer 204. In various implementations, the manifold layer 206 may be formed from a sheet of polyurethane, such as a vacuum-formed sheet of polyurethane having a thickness of about 0.5 millimeters. In various implementations, the manifold layer 206 may be formed from a polymer material that is substantially clear or optically transparent, allowing the user to see through the manifold layer 206.

In various implementations, windows 236 may be removed from the manifold layer 206 and form a grid pattern. For example, the plurality of windows 236 may be arranged in a pattern of rows and columns. The center of each window 236 may be aligned with the center of each other window 236 within a row, and the center of each window 236 may be aligned with the center of each other window 236 within a column. In various implementations, a plurality of standoffs 238 may be formed on the bottom surface 230 of the manifold layer 206. In various implementations, the plurality of standoffs 238 may form a grid pattern. For example, the plurality of standoffs 238 may be arranged in a pattern of rows and columns. The center of each standoff 238 may be aligned with the center of each other standoff 238 within a row, and the center of each standoff 238 may be aligned with the center of each other standoff 238 within a column. In various implementations, each row of the plurality of windows 236 may be disposed adjacent to a row of the plurality of standoffs 238, and each column of the plurality of windows 236 may be disposed adjacent to a column of the plurality of standoffs 238. In various implementations, the plurality of windows 236 and the plurality of standoffs 238 may be arranged in a pattern such that rows of the pattern alternate between rows of the plurality of windows 236 and rows of the plurality of standoffs 238, and columns of the pattern alternate between columns of the plurality of windows 236 and columns of the plurality of standoffs 238.

In various implementations, each window 236 may be substantially circular in profile in the plane of the top surface 230 of the manifold layer 206. In various implementations, each standoff 238 may be substantially circular in profile and protrude outwardly in a substantially orthogonal manner from the plane of the bottom surface 230 of the manifold layer 206. In various implementations, a diameter of each window 236 may be greater than a diameter of each standoff 238. For example, each window 236 may have a diameter of about eight millimeters, and each standoff 238 may have a diameter of about three millimeters. In various implementations, each standoff 238 may have a height of in a range of about 0.5 millimeters to about 3 millimeters. In various implementations, each standoff 238 may have a height of about 2.5 millimeters. In various implementations, each standoff 238 may have a height of about 3 millimeters. In various implementations, each standoff 238 within a row may be spaced a distance of about four millimeters on center from an adjacent standoff 238 within a row, and each standoff 238 within a column may be spaced a distance of about four millimeters on center from an adjacent standoff 238 within a column. In various implementations, the plurality of standoffs 238 may be right cylinders with hemispherical ends, such as half-capsules, and may be formed on and protrude substantially away from the bottom surface 230 of the manifold layer 206 in a direction substantially normal to the bottom surface 230. In various implementations, each of the plurality of standoffs 238 may have a height in a range of about 2.5 millimeters to about three millimeters.

As shown in FIG. 2, examples of the manifold layer 206 may include a raised portion, such as a lip portion or a boss 240. In various implementations, the boss 240 may protrude outwardly in a substantially orthogonal manner from the plane of the top surface 232 of the manifold layer 206. In various implementations, the boss 240 may have a scaled down profile or outline similar to the shape of the periphery 234. In various implementations, the manifold layer 206 may also have a border region 242 between the boss 240 and the periphery 234, and the border region 242 may not include any windows 236 or standoffs 238. The manifold layer 206 may also include an opening, such as aperture 239, and a region surrounding the aperture 239 that does not have any windows 236 or standoffs 238, such as coupling region 243.

The second film layer 208 may have a first side or bottom surface 244 opposite a second side or top surface 246, and a periphery 248 defined by a perimeter of the second film layer 208. The bottom surface 244 of the second film layer 208 may be disposed adjacent to the top surface 232 of the manifold layer 206. A negative-pressure aperture, such as aperture 250, may be formed through the second film layer 208. In various implementations, the second film layer 208 may be formed from or include any of the materials previously described with respect to the cover 110 and/or the first film layer 204.

As shown in FIG. 2, examples of the dressing 104 may include a cover 110 having a top surface 254 opposite a bottom surface 256, and a periphery 258 defined by an outer perimeter of the cover 110. A central aperture 260 may be formed through the cover 110. In some examples, the second film layer 208 may form a portion of the cover 110 by being coupled to the cover 110 and exposed through the central aperture 260, or the second film layer 208 may be integrally formed with the cover 110 as a central portion of the cover 110.

In various implementations, the periphery 214 of the sealing layer 202 may be substantially coextensive with the periphery 258 of the cover 110. In various implementations, the periphery 224 of the first film layer 204, the periphery 234 of the manifold layer 206, and the periphery 248 of the second film layer 208 may be substantially coextensive. In various implementations, the outline of the treatment aperture 216 of the sealing layer 202 may be substantially coextensive with the outline of the central aperture 260 of the cover 110. In various implementations, the outlines of the treatment aperture 216 and the central aperture 260 may be substantially similar to the outlines of the periphery 224, the periphery 234, and the periphery 248. In various implementations, the outlines of the treatment aperture 216 and the central aperture 260 may be substantially similar to but scaled down from the outlines of the periphery 224, the periphery 234, and the periphery 248. In assembled form, the sealing layer 202, the first film layer 204, the manifold layer 206, the second film layer 208, and the cover 110 may be stacked such that the periphery 214 is aligned with the periphery 258, and the periphery 224 is aligned with the periphery 234 and the periphery 248. In various implementations, the treatment aperture 216 may be aligned with the central aperture 260, and the periphery 224, the periphery 234, and the periphery 248 are positioned such that they are aligned with and evenly extend past the outlines of the treatment aperture 216 and the central aperture 260.

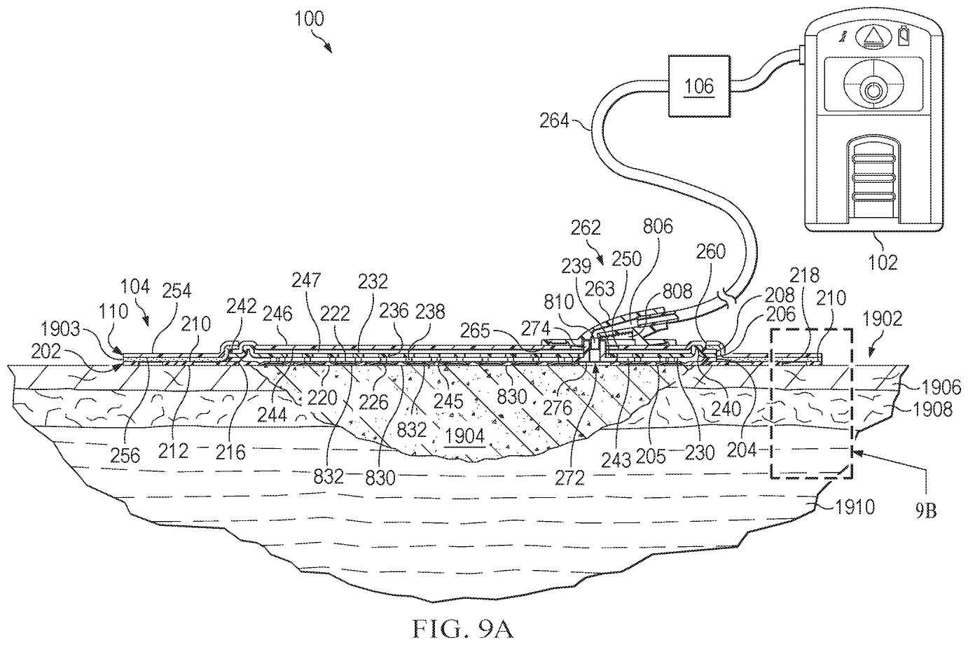

In various implementations, a portion of the top surface 222 of the first film layer 204 near the periphery 224 may be coupled to a portion of the bottom surface 230 of the manifold layer 206 at the border region 242 to define a first chamber 245, shown in FIG. 9A, between the first film layer 204 and the manifold layer 206. In various implementations, a portion of the bottom surface 244 of the second film layer 208 near the periphery 248 may be coupled to a portion of the top surface 232 of the manifold layer 206 at the border region 242 to define a second chamber 247, shown in FIG. 9A, between the second film layer 208 and the manifold layer 206. In various implementations, aperture 239 of the manifold layer 206, and aperture 250 of the second film layer 208 may be substantially coaxially aligned.

In various implementations, a portion of the top surface 210 of the sealing layer 202 around the treatment aperture 216 may be coupled to a portion of the bottom surface 220 of the first film layer 204 near the periphery 224, and a portion of the bottom surface 256 of the cover 110 around the central aperture 260 may be coupled to a portion of the top surface 246 of the second film layer 208 near the periphery 248. In various implementations, a portion of the top surface 210 of the sealing layer 202 between the periphery 214 and the treatment aperture 216 may be coupled to a portion of the bottom surface 256 of the cover 110 between the periphery 258 and the central aperture 260.

Some examples of the dressing 104 also include a dressing interface 262 and a fluid conductor 264. In various implementations, the fluid conductor 264 may be a flexible tube that can be fluidly coupled on one end to the dressing interface 262. In various implementations, the dressing interface 262 may be an elbow connector that can be placed over the aperture 250 to provide a fluid path between the fluid conductor 264 and the interior of the dressing 104. For example, the dressing interface 262 may be coupled to the top surface 246 of the second film layer 208 over the aperture 250. In some examples, the second film layer 208 may be formed integrally with the cover 110 as a unified or single layer of material such that the second film layer 208 forms a central portion of the cover 110. Further, in some examples, the second film layer 208 may be referred to herein as a cover regardless of whether the second film layer 208 is formed integrally with the cover 110. In some examples, the dressing interface 262 may include a housing 263, and the housing 263 may include a mounting surface 265 configured to be coupled to a cover or a first portion of the dressing 104, such as the second film layer 208, around the aperture 250.

Further, some examples of the dressing 104 may include a sensing conduit 272 configured to be coupled between a portion of the housing 263 of the dressing interface 262 and the tissue contact surface 205 of the tissue interface 108. The sensing conduit 272 may include a first end 274 in fluid communication with a second end 276 through the sensing conduit 272. The first end 274 of the sensing conduit 272 is configured to be fluidly coupled to a portion of the housing 263, and the second end 276 of the sensing conduit 272 is configured to be positioned proximate to the tissue contact surface 205 of the tissue interface 108. The sensing conduit 272 is configured to pass through at least a portion of the thickness of the tissue interface 108, such as, for example, the thickness of the manifold 206 between the top surface 232 and the bottom surface 230, and the thickness of the second film layer 208 between the top surface 246 and the bottom surface 244 as shown in FIG. 2.

As illustrated in FIG. 2, some examples of the dressing 104 may include a release liner 268 to protect the sealing layer 202 and the adhesive coated on the bottom surface 256 of the cover 110 prior to use. The release liner 268 may also provide stiffness to assist with, for example, deployment of the dressing 104. In various implementations, the release liner may include a polyethylene terephthalate (PET) or similar polar semi-crystalline polymer. The use of a polar semi-crystalline polymer for the release liner 268 may substantially preclude wrinkling or other deformation of the dressing 104. The polar semi-crystalline polymer may be highly orientated and resistant to softening, swelling, or other deformation that may occur when objects are brought into contact with the layers and/or components of the dressing 104, or when the dressing 104 is subjected to temperature or environmental variations, or during sterilization. Further, a release agent may be disposed on a top surface 270 of the release liner 268 that is configured to contact the bottom surface 212 of the sealing layer 202 and the adhesive disposed on the bottom surface 256 of the cover 110. For example, the release agent may be a silicone coating and may have a release factor suitable to facilitate removal of the release liner 268 by hand and without damaging or deforming the dressing 104. In various implementations, the release agent may be a fluorocarbon or a fluorosilicone. In various implementations, the release liner 268 may be uncoated or otherwise used without a release agent.

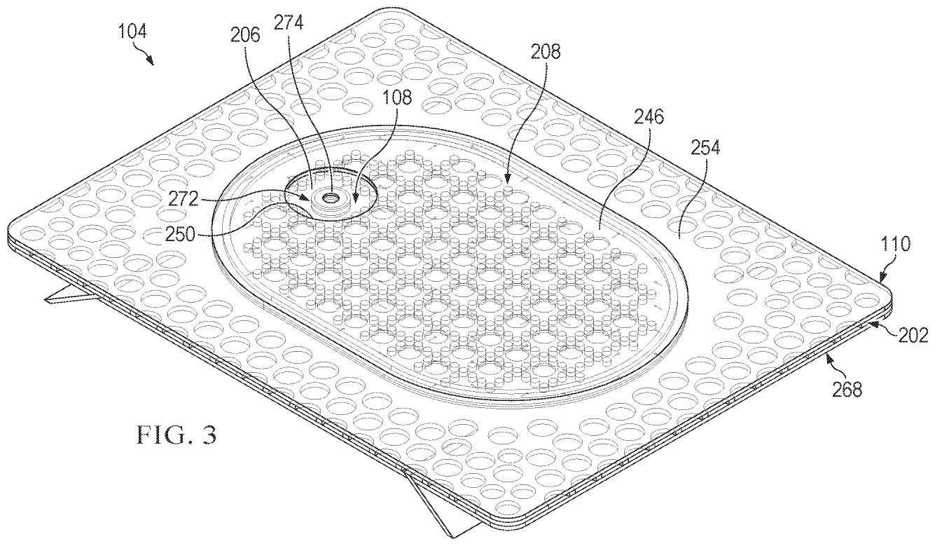

FIG. 3 is an isometric view of an assembled example of the dressing 104 of FIG. 2. As shown in FIG. 3, in some examples, the cover 110, the second film layer 208, the manifold layer 206, and/or the first film layer 204 may be substantially clear or optically transparent, allowing for visualization of the layers of the dressing 104 as well as visualization through the windows 236 of the manifold layer 206.

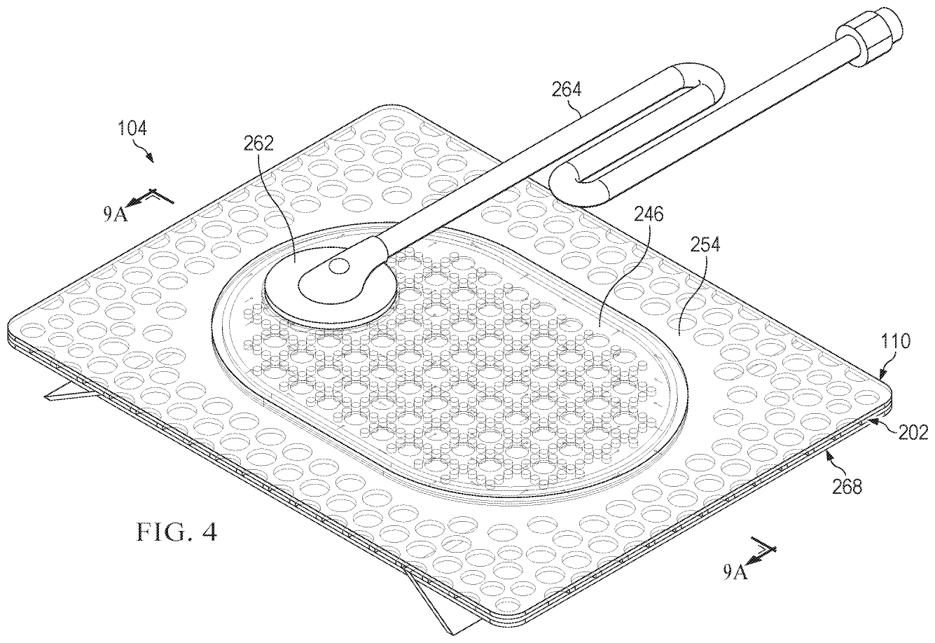

FIG. 4 is an isometric view of the assembled dressing 104 of FIGS. 2 and 3 with dressing interface 262 attached. As shown in FIG. 4 with reference to FIG. 3, in some examples, the dressing interface 262 and the fluid conductor 264 may be attached to the top surface 246 and/or top surface 254 and fluidly coupled to the tissue interface 108 within the interior of the dressing 104 via the aperture 250. In various implementations, the fluid conductor 264 may be connected to the negative-pressure source 102.

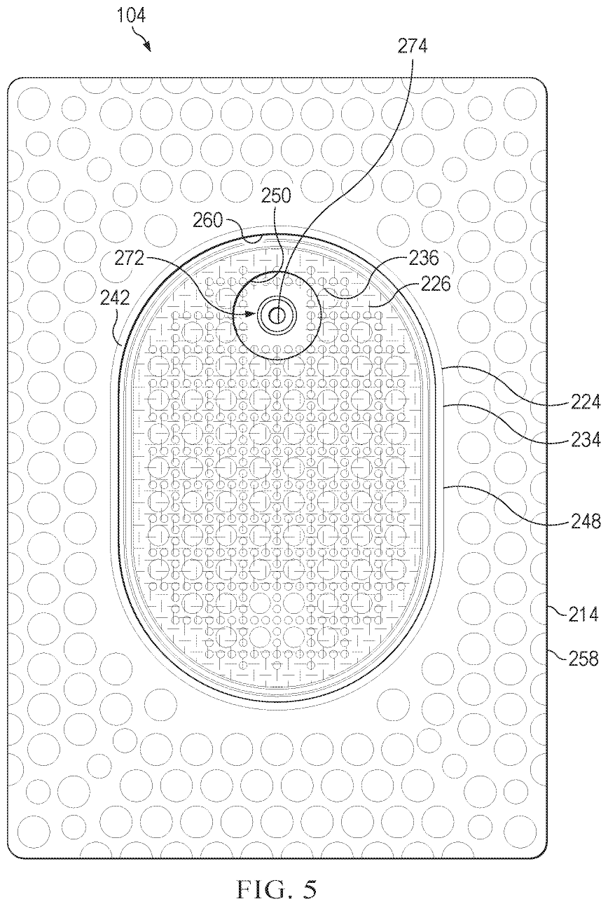

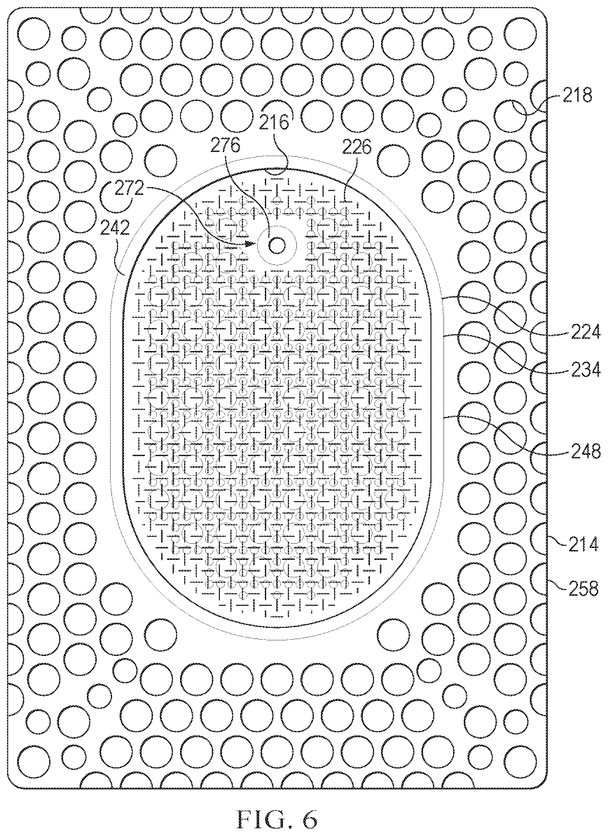

FIG. 5 is a top view of the dressing 104 of FIGS. 2-4, as assembled, illustrating details that may be associated with some examples. FIG. 6 is a bottom view of the dressing 104 of FIGS. 2-5, illustrating details that may be associated with some examples. As illustrated in FIGS. 5 and 6, in some examples of the dressing 104, the periphery 258 of the cover 110 may be coextensive with the periphery 214 of the sealing layer 202. In various implementations, the periphery 224 of the first film layer 204 may be coextensive with the periphery 234 of the manifold layer 206 and the periphery 248 of the second film layer 208. In various implementations, the perimeter or outline of the central aperture 260 of the cover 110 may be coextensive with the perimeter or outline of the treatment aperture 216 of the sealing layer 202 in a plane defined by the top surface 254 of the cover 110 or the bottom surface 212 of the sealing layer 202.

As previously described with reference to FIG. 2, the perimeters or outlines of the treatment aperture 216 and the central aperture 260 may be similar in shape to the perimeters or outlines of the periphery 224, periphery 234, and periphery 248, but scaled down such that in assembled form, a portion of the sealing layer 202 surrounding the treatment aperture 216 overlaps with a portion of the first film layer 204 around the periphery 224, and a portion of the cover 110 surrounding the central aperture 260 overlaps with a portion of the second film layer 208 around the periphery 248, for example, at the border region 242.

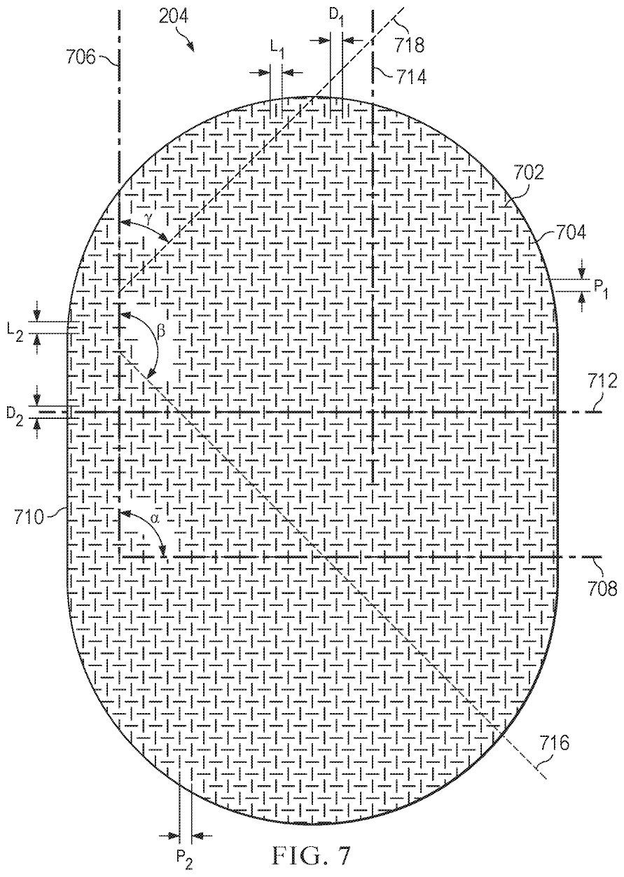

FIG. 7 is a top view illustrating additional details that may be associated with some examples of the first film layer 204. For example, as illustrated in FIG. 7, the fluid passages 226 may include a first plurality of perforations 702 and a second plurality of perforations 704. Each of the first plurality of perforations 702 and the second plurality of perforations 704 may be linear or curved perforations, such as slots or slits. In some embodiments where the perforations are linear slots or slits, each of the first plurality of perforations 702 may have a length Ly and each of the second plurality of perforations 704 may have a length L₂. In some embodiments, where the perforations are curved slots or slits, each of the first plurality of perforations may have a length L measured from an end of the curved slot or slit to the other end of the curved slot or slit, and each of the second plurality of perforations may have a length L₂ measured from an end of the curved slot or slit to the other end of the curved slot or slit. In some embodiments, the length L₁ may be equal to the length L₂. The first plurality of perforations 702 and the second plurality of perforations 704 may be distributed across the second layer in one or more rows in one direction or in different directions.

In example embodiments, each of the first plurality of perforations 702 may have a first long axis. In some embodiments, the first long axis may be parallel to a first reference line 706 running in a first direction. In illustrative examples, each of the second plurality of perforations 704 may have a second long axis. In example embodiments, the second long axis may be parallel to a second reference line 708 running in a second direction. In some embodiments, one or both of the first reference line 706 and the second reference line 708 may be defined relative to an edge 710 or line of symmetry of the first film layer 204. For example, one or both of the first reference line 706 and the second reference line 708 may be parallel or coincident with an edge 710 or line of symmetry of the first film layer 204. In some illustrative embodiments, one or both of the first reference line 706 and the second reference line 708 may be rotated an angle relative to an edge 710 of the first film layer 204. In example embodiments, an angle α may define the angle between the first reference line 706 and the second reference line 708.

In some example embodiments, the centroid of each of the first plurality of perforations 702 within a row may intersect a third reference line 712 running in a third direction. In illustrative embodiments, the centroid of each of the second plurality of perforations 704 within a row may intersect a fourth reference line 714 running in a fourth direction. In general, a centroid refers to the center of mass of a geometric object. In the case of a substantially two dimensional object such as a linear slit, the centroid of the linear slit will be the midpoint.

The pattern of fluid passages 226 may also be characterized by a pitch, which indicates the spacing between corresponding points on fluid passages 226 within a pattern. In example embodiments, pitch may indicate the spacing between the centroids of fluid passages 226 within the pattern. Some patterns may be characterized by a single pitch value, while others may be characterized by at least two pitch values. For example, if the spacing between centroids of the fluid passages 226 is the same in all orientations, the pitch may be characterized by a single value indicating the spacing between centroids in adjacent rows. In example embodiments, a pattern comprising a first plurality of perforations 702 and a second plurality of perforations 704 may be characterized by two pitch values, P₁ and P₂, where P₁ is the spacing between the centroids of each of the first plurality of perforations 702 in adjacent rows, and P₂ is the spacing between the centroids of each of the second plurality of perforations 704 in adjacent rows.

In example embodiments, within each row of the first plurality of perforations 702, each perforation may be separated from an adjacent perforation by a distance D₁. In some embodiments, within each row of the second plurality of perforations 704, each perforation may be separated from an adjacent perforation by a distance D₂. In some patterns, the rows may be staggered. The stagger may be characterized by an orientation of corresponding points in successive rows relative to an edge or other reference line associated with the first film layer 204. In some embodiments, the rows of the first plurality of perforations 702 may be staggered. For example, a fifth reference line 716 in a fifth direction runs through the centroids of corresponding perforations of adjacent rows of the first plurality of perforations 702. In some example embodiments, the stagger of the rows of the first plurality of perforations 702 may be characterized by the angle β formed between the first reference line 706 and the fifth reference line 716. In additional illustrative embodiments, the rows of the second plurality of perforations 704 may also be staggered. For example, a sixth reference line 718 in a sixth direction runs through the centroids of corresponding perforations of adjacent rows of the second plurality of perforations 704. In some embodiments, the stagger of the rows of the second plurality of perforations 704 may be characterized by the angle γ formed between the first reference line 706 and the sixth reference line 718.

FIG. 7 illustrates an example of a pattern that may be associated with some embodiments of the fluid passages 226. In the example of FIG. 7, each of the first plurality of perforations 702 and the second plurality of perforations 704 may be linear slots or slits. The first reference line 706 may be parallel with an edge 710, and the second reference line 708 may be orthogonal to the edge 710. In example embodiments, the third reference line 712 is orthogonal to the first reference line 706, and the fourth reference line 714 is orthogonal to the second reference line 708. For example, the third reference line 712 may be incident with the centroids of corresponding perforations in alternating rows of the second plurality of perforations 704, and the fourth reference line 714 may intersect the centroids of corresponding perforations in alternating rows of the first plurality of perforations 702. In the example of FIG. 7, the fluid passages 226 are arranged in a cross-pitch pattern in which each of the first plurality of perforations 702 is orthogonal along its first long axis to each of the second plurality of perforations 704 along its second long axis. For example, in FIG. 7, P₁ is equal to P₂ (within acceptable manufacturing tolerances), and the cross-pitch pattern may be characterized by a single pitch value. Additionally, L₁ and L₂ may be substantially equal, and D₁ and D₂ may be also be substantially equal, all within acceptable manufacturing tolerances. The rows of the first plurality of perforations 702 and the rows of the second plurality of perforations 704 may be characterized as staggered. For example, in some example embodiments illustrated, a may be about 90°, β may be about 135°, γ may be about 45°, P₁ may be about 4 mm, P₂ may be about 4 mm, L₁ may be about 3 mm, L₂ may be about 3 mm, D₁ may be about 5 mm, and D₂ may be about 5 mm.

In additional embodiments, P/may be in a range of about 4 millimeters to about 6 millimeters, P₂ may be in a range of about 3 mm to about 6 mm. In illustrative embodiments, D₁ may be in a range of about 3 mm to about 5 mm, and D₂ may be in a range of about 3 mm to 5 mm. In some embodiments, there may be an equal number of fluid passages 226 in the first plurality of perforations 702 as the number of fluid passages 226 in the second plurality of perforations 704. Although FIG. 7 illustrates an example pattern for the fluid passages 226 of the first film layer 204, other patterns are possible, such as the pattern of the fluid passages 226 illustrated in FIGS. 2-4.

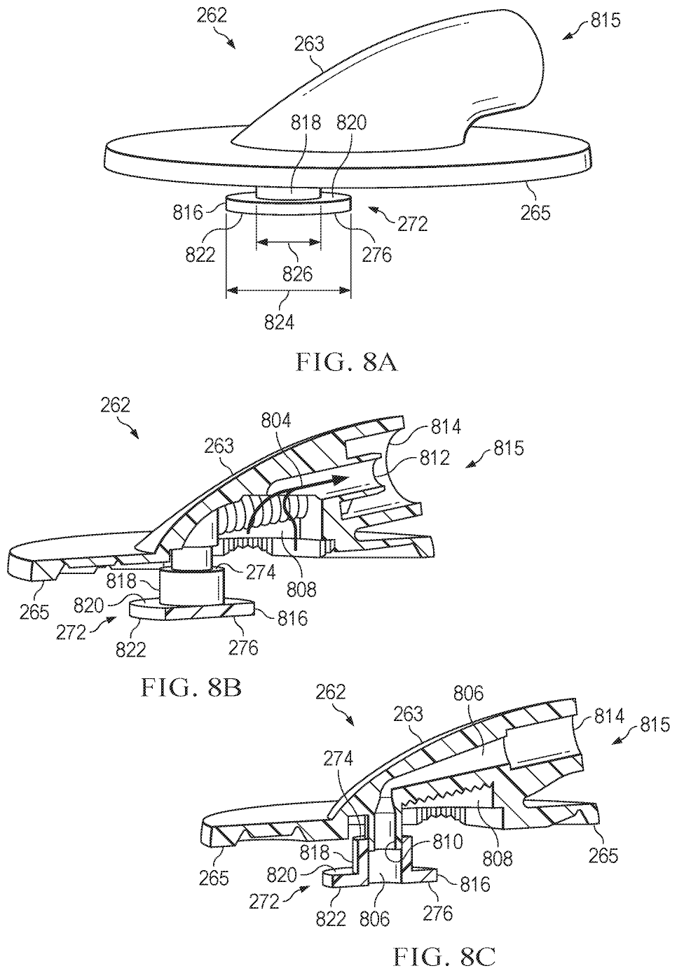

FIGS. 8A-8C are isometric views of illustrative examples of the dressing interface 262 and the sensing conduit 272, illustrating additional details that may be associated with some examples. The dressing interface 262 may include the housing 263, a fluid pathway 804 shown in FIG. 8B, and a sensing pathway 806 shown in FIG. 8C. In some examples, the housing 263 may include a fluid inlet cavity 808 shown in FIGS. 8B and 8C, and a sensing inlet port 810 shown in FIG. 8C. In some examples, the mounting surface 265 of the housing 263 may surround the fluid inlet cavity 808 and the sensing inlet port 810.

FIG. 8B is a cross-sectional view of the dressing interface 262 taken through the fluid pathway 804, illustrating the fluid pathway 804 passing internally through the housing 263 and in fluid communication with the fluid inlet cavity 808 proximate to the mounting surface 265. In some examples, the fluid pathway 804 may extend internally through the housing 263 between the fluid inlet cavity 808 and a fluid outlet port 812 external to the housing 263.

FIG. 8C is a cross-sectional view of the dressing interface 262 taken through the sensing pathway 806, illustrating the sensing pathway 806 passing internally through the housing 263 and in fluid communication with the sensing inlet port 810. In some examples, the sensing pathway 806 may extend internally through the housing 263 between the sensing inlet port 810 and a sensing outlet port 814 external to the housing 263. The sensing pathway 806 shown in FIG. 8C may be fluidly isolated from the fluid pathway 804 shown in FIG. 8B. The sensing conduit 272 may be configured to be coupled between the sensing inlet port 810 and the tissue contact surface 205 of the tissue interface 108, shown in FIGS. 2 and 9A. The housing 263 can further include at least one fluid conductor connection 815 configured to connect a fluid conductor, such as the fluid conductor 264, to each of the fluid outlet port 812 and the sensing outlet port 814.

Continuing with FIGS. 8A-8C and with reference to FIGS. 2 and 9A, in some examples, the fluid inlet cavity 808 and the sensing inlet port 810 can be configured to be exposed to the tissue interface 108 through the aperture 250. Further, the first end 274 of the sensing conduit 272 can be configured to be fluidly coupled to the sensing inlet port 810, and the second end 276 of the sensing conduit 272 can be configured to be positioned proximate to the tissue contact surface 205 of the tissue interface 108. In some examples, the sensing conduit 272 can be in fluid communication between the sensing pathway 806 and the tissue contact surface 205 such that the sensing pathway 806 extends through the sensing conduit 272 fluidly isolated from the fluid pathway 804.

In some examples, the second end 276 of the sensing conduit 272 can include a flange 816 that extends outward from an exterior surface 818 of the sensing conduit 272. The flange can include a first surface 820 and a second surface 822 opposite the first surface 820. The first surface 820 can be configured to face the mounting surface 265 of the dressing interface 262, and the second surface 822 can be configured to face a tissue site. The flange 816 can extend outward, for example, perpendicular to the exterior surface 818 of the sensing conduit 272. The flange 816 can include an external diameter 824 that is larger than an external diameter 826 of the exterior surface 818 of the sensing conduit 272. Further, in some examples, the mounting surface 265 can be configured to be coupled to a cover, such as the cover 110 or the top surface 246 of the second film layer 208. A portion of the tissue interface 108, such as, for example, the manifold 206, can be configured to be captured between the cover and the first surface 820 of the flange 816 of the sensing conduit 272.

The second end 276 of the sensing conduit 272 and/or the flange 216 can be positioned proximate to, on, or at the tissue contact surface 205 of the tissue interface 108 in a variety of ways. In some examples, the first surface 820 of the flange 816 can be configured to contact the tissue contact surface 205 of the tissue interface 108. In some examples, the second surface 822 of the flange 816 can be configured to contact a tissue site. In some examples, the flange 816 can be coupled to the tissue contact surface 205 of the tissue interface 108. In some examples, the flange 816 can be formed integrally with the tissue contact surface 205 of the tissue interface 108. In some examples, the flange 816 of the sensing conduit 272 can be positioned on the second side or top surface 222 of the first film layer 204 with the sensing conduit 272 being in fluid communication with the first side or bottom surface 220 of the first film layer 204 through at least one of the plurality of fluid passages 226 or another aperture (not shown) through the first film layer 204. In some examples, the flange 816 of the sensing conduit 272 can be positioned on the first side or bottom surface 220 of the first film layer 204. In some examples, the flange 816 of the sensing conduit 272 can be configured to be positioned between the first film layer 204 and the manifold 206.

Although FIGS. 8A-8C illustrate a configuration for the mounting surface 265, the inlet cavity 808, and the sensing inlet port 810 that may be advantageous for use with the dressing 104, these and other features of the dressing interface 262 may be simplified as desired, for example, for other types of dressings. Such a simplified example of the dressing interface 262 can include the housing 263 and the sensing conduit 272 configured to pass into or through a thickness of the dressing 104, or another dressing, for coupling to a portion of the housing 263. The housing 263 can be configured to be coupled, in any suitable manner, to a first portion of a dressing, such as the second film layer 208, the cover 110, or another cover structure defining an exterior surface of the dressing 104 or another dressing. The housing 263 can include the fluid pathway 804 and the sensing pathway 806. The sensing conduit 272 can include the first end 274 configured to be coupled, in any suitable manner, to the sensing pathway 806. The second end 276 of the sensing conduit 272 can also be configured in any suitable manner to be coupled to a second portion of a dressing, such as a portion of the dressing 104 proximate to or on the tissue contact surface 205 of the tissue interface 108. For example, the second end 276 of the sensing conduit 272 can include the flange 816 configured to be coupled to the second portion of the dressing 104, or to an analogous portion of another dressing. Additionally or alternatively, the second end 276 of the sensing conduit 272 can be formed integrally with the tissue contact surface 205 of the tissue interface 108. For example, the second end 276 of the sensing conduit 272 can be molded into or as part of the tissue contact surface 205. The sensing pathway 806 can extend through the sensing conduit 272 to the second portion of the dressing 104, or another dressing by way of analogy, with the sensing pathway 806 being fluidly isolated from the fluid pathway 804.

In some examples, a system for treating a tissue site, such as the system 100 previously introduced in FIG. 1, can include the negative-pressure source 102 configured to be fluidly coupled to the fluid pathway 804, and a pressure sensor, such as the first sensor 114, configured to be fluidly coupled to the sensing pathway 806. An example of such a system is illustrated in FIG. 9A. FIG. 9A is a cross-sectional view of the example dressing 104 of FIG. 4, taken at line 9A-9A, applied to an example tissue site 1902, and illustrating additional details associated with the therapy system 100 of FIG. 1. As shown in FIG. 9A, in some examples, the tissue contact surface 205 of the tissue interface 108 can be configured to be positioned in direct contact with a tissue site. Further, in some examples, the tissue contact surface 205 of the tissue interface 108 can include a pattern or plurality of surface features 830 that outwardly project from the tissue contact surface 205 and are configured to define a fluid communication space 832 between a tissue site and the tissue contact surface 205. The fluid communication space 832 can be a space or plurality of spaces between and/or among the surface features 830 that forms a pattern in cooperation with the surface features 830.

Further, as shown in FIG. 9A, in various implementations, the bottom surface 256 of the cover 110 may be coated with an adhesive layer 1903, and at least a portion of the cover 110 may be coupled to at least a portion of the top surface 210 of the sealing layer 202 with the adhesive layer 1903. The adhesive layer 1903 may be any of the attachment devices previously discussed with reference to FIG. 1. At least a portion of the bottom surface 256 of the cover 110 may be coupled to at least a portion of the top surface 246 of the second film layer 208, for example, at the border region 242, by the adhesive layer 1903. In various implementations, at least a portion of the bottom surface 220 of the first film layer 204, for example, at the border region 242, may be coupled to at least a portion of the top surface 210 of the sealing layer 202.

In some examples, the dressing 104 may be applied to the tissue site 1902 and cover a wound 1904. The tissue site 1902 may be or may include a defect or targeted treatment site, such as the wound 1904, which may be partially or completely filled or covered by the dressing 104. In some examples, the wound 1904 may be in epidermis 1906. In some examples, the wound 1904 may extend through the epidermis 1906 and into a dermis 1908. In some examples, as shown in FIG. 9A, the wound 1904 may extend through the epidermis 1906 and dermis 1908 into a subcutaneous tissue 1910. In some applications, at least a portion of the bottom surface 212 of the sealing layer 202 may be brought into contact with a portion of the epidermis 1906 surrounding the wound 1904, and at least a portion of the bottom surface 220 of the first film layer 204 may be placed within, over, on, against, or otherwise proximate to the wound 1904. The bottom surface 220 of the first film layer 204 may also be the tissue contact surface 205 in some examples.

In operation, negative pressure may be provided to the wound 1904, and/or fluid may be removed from the wound 1904 by the negative-pressure source 102. For example, fluid may travel from the wound 1904 through at least one of the fluid passages 226 into the first chamber 245 between the top surface 222 of the first film layer 204 and the bottom surface 230 of the manifold layer 206. The fluid may then travel through the windows 236 of the manifold layer 206 and into the second chamber 247 between the top surface 232 of the manifold layer 206 and the bottom surface 244 of the second film layer 208. The fluid may then travel through the aperture 250 and into the dressing interface 262, and from the dressing interface 262 to the negative-pressure source 102 through the fluid conductor 264 and/or the container 106.

In operation, a pressure feedback signal corresponding to the negative pressure present at the tissue contact surface 205 in contact with the wound 1904 may travel through the sensing conduit 272 and the sensing pathway 806 back to the controller 112 and/or a pressure sensor, such as the sensor 114. The pressure feedback signal may travel through a fluid conductor, such as a portion the fluid conductor 264 that is fluidly isolated from the negative pressure communicated to the wound 1904, or another dedicated fluid conductor 264. In such a configuration, the sensing pathway 806 is fluidly isolated from the fluid pathway 804 and interior spaces of the dressing 104, such as the manifold 206, the first chamber 245, and the second chamber 247, providing a direct path to the wound 1904 with minimal pressure drop or other losses. In this manner, the accuracy of the pressure feedback signal can be improved.

FIG. 9B is a detail view, taken at reference 9B in FIG. 9A, illustrating details that may be associated with some example embodiments of the example dressing 104 of FIG. 9A. In various implementations, the sealing layer 202 may be sufficiently tacky at the bottom surface 212 to hold the dressing 104 in position relative to the epidermis 1906 and/or the wound 1904, while allowing the dressing 104 to be removed or repositioned without trauma to the tissue site 1902. In various implementations, the sealing layer 202 may be formed of a silicone material, which may form sealing couplings at the bottom surface 212 with the epidermis 1906. In various implementations, the bond strength or tackiness of the sealing couplings may have a peel adhesion or resistance to being peeled from a stainless steel material between about 0.5 N/25 mm to about 1.5 N/25 mm on stainless steel substrate at about 25° C. at about 50% relative humidity based on ASTM D3330. The sealing layer 202 may achieve this bond strength after a contact time of less than about 60 seconds. Tackiness may be considered a bond strength of an adhesive after a very low contact time between the adhesive and a substrate. In various implementations, the sealing layer 202 may have a thickness in a range of about 200 micrometers to about 1,000 micrometers. Removing the release liner 268 may also expose adhesive layer 1903 through the apertures 218 of the sealing layer 202. In the assembled state, the thickness of the sealing layer 202 may create a gap between the adhesive layer 1903 and the epidermis 1906 through the apertures 218 of the scaling layer 202 such that the adhesive 1903 is not in contact with the epidermis 1906.

FIG. 9C is a detail view illustrating additional details that may be associated with the detail view of FIG. 9B in some implementations of the dressing 104 of FIG. 9A. FIG. 9C illustrates the adhesive layer 1903 after a portion of it has been brought into contact with the epidermis 1906 by a force vector 1912 applied to the top surface 254 of the cover 110 at the apertures 218. In use, if the assembled dressing 104 is at the desired location, the force vector 1912 may be applied to the top surface 254 at the apertures 218 to cause at least a portion of the adhesive layer 1903 to be pressed at least partially into contact with the epidermis 1906 through at least one or more of the apertures 218 to form bonding couplings. The bonding couplings may provide secure, releasable mechanical fixation of the dressing 104 to the epidermis 1906. In various implementations, the sealing couplings may not be as mechanically strong as the bonding couplings. The bonding couplings may anchor the dressing 104 to the epidermis 1906, inhibiting and/or substantially preventing migration of the dressing 104.

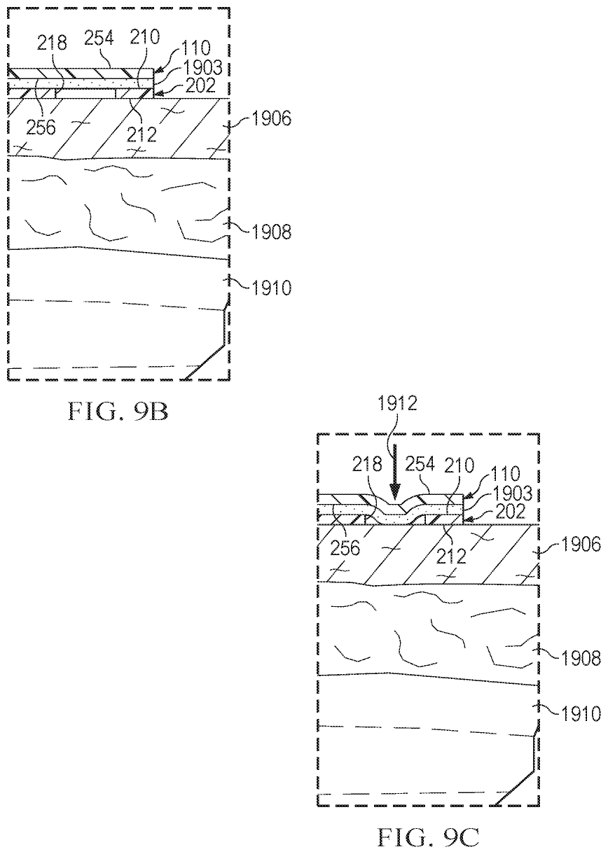

FIG. 10 is a detail view of an example of the dressing 104, the dressing interface 262, and the sensing conduit 272 positioned at the tissue site 1902 according to this specification, illustrating additional details that can be associated with some previously described example embodiments.

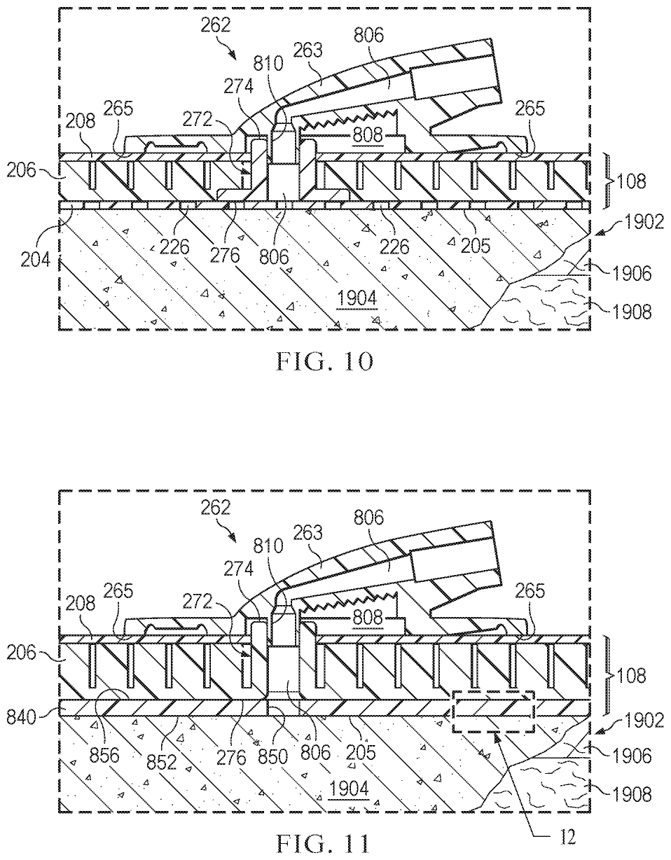

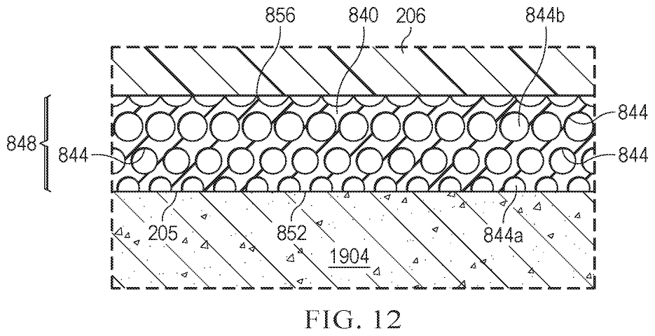

FIG. 11 is a detail view of another example of the dressing 104, the dressing interface 262, and the sensing conduit 272 positioned at the tissue site 1902 according to this specification, illustrating additional details that can be associated with some example embodiments. FIG. 12 is a detail view of the example dressing 104, the dressing interface 262, and the sensing conduit 272 of FIG. 11, illustrating additional details that can be associated with some example embodiments. Referring collectively to FIGS. 11 and 12, in some examples, the sensing conduit 272 may be molded or otherwise formed integrally into or as a portion of the manifold layer 206. Further, in some examples, at least a portion of the tissue contact surface 205 can include a porous layer 840 including a plurality of interconnected pores 844 that have a variable or graded porosity and are configured to provide fluid flow through a thickness 848 of the porous layer 840 between the wound 1904 and the manifold 206. For example, the plurality of interconnected pores 844 can increase in size through the thickness 848 of the porous layer 840 from a first side 852 of the porous layer 840 to a second side 856 of the porous layer 840 that is opposite the first side 852. For example, a first pore 844a proximate to or at the first side 852 can be smaller than a second pore 844b proximate to or at the second side 856. Further, the circular shape and pattern of the interconnected pores 844 in FIG. 12 are provided solely for illustration. The interconnected pores 844 can have any suitable shape and pattern and can include broken or open cell walls (not shown) through the thickness 848 that provide fluid communication and interconnection among the interconnected pores 844. The first side 852 of the porous layer 840 can be configured to contact a tissue site, such as the wound 1904. The second end 276 of the sensing conduit 272 can be positioned at the second side 856 of the porous layer 840. An aperture 850 can be disposed through the thickness 848 and the first side 852 and the second side 854 of the porous layer 840. The aperture 850 can be aligned with the second end 276 of the sensing conduit 272 and the sensing pathway 806 to position the sensing pathway 806 in direct fluid communication with the wound 1904. In such a configuration, the porous layer 840 may provide cushioning between the wound 1904 and the sensing conduit 272 while the positioning of the smaller interconnected pores 844a proximate to or at the first side 852 in contact with the tissue site can reduce tissue in-growth from the wound 1904. Further, the porous layer 840 and the interconnected pores 844 can define a fluid communication space underneath the manifold 206 and at the tissue contact surface 205, for example, to provide lateral fluid communication in a direction orthogonal to the thickness 848 of the porous layer 840, which can enhance the pressure feedback signal through the sensing pathway 806 in some examples. The porous layer 840 can also be used in various example embodiments of the dressing 104 in addition to or in lieu of the first film layer 204. Further, in some examples, the porous layer 840 may include or may be an aerogel material.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing configured to be positioned adjacent to a tissue site, comprising:
a cover configured to create a seal at the tissue site;
a tissue interface including a tissue contact surface configured to be positioned in contact with the tissue site;
a dressing interface configured to be coupled to the cover, the dressing interface comprising:
a housing including a mounting surface surrounding a fluid inlet cavity and a sensing inlet port,
a fluid pathway extending internally through the housing between the fluid inlet cavity and a fluid outlet port external to the housing, and
a sensing pathway extending internally through the housing between the sensing inlet port and a sensing outlet port external to the housing, the sensing pathway fluidly isolated from the fluid pathway; and
a sensing conduit configured to be coupled between the sensing inlet port and the tissue contact surface of the tissue interface.

2. The dressing of claim 1, wherein the mounting surface is configured to be coupled to the cover, and wherein the fluid inlet cavity and the sensing inlet port are configured to be exposed to the tissue interface through an aperture in the cover.

3. The dressing of claim 1, wherein the sensing conduit comprises a first end in fluid communication with a second end through the sensing conduit, the first end configured to be fluidly coupled to the sensing inlet port and the second end configured to be positioned proximate to the tissue contact surface of the tissue interface.

4. The dressing of claim 1, wherein the sensing conduit is in fluid communication between the sensing pathway and the tissue contact surface such that the sensing pathway extends through the sensing conduit fluidly isolated from the fluid pathway.

5. The dressing of claim 1, wherein the sensing conduit passes through at least a portion of a thickness of the tissue interface.

6. The dressing of claim 3, wherein the second end of the sensing conduit includes a flange that extends outward from an exterior surface of the sensing conduit, wherein the flange includes a first surface and a second surface opposite the first surface, the first surface configured to face the mounting surface of the dressing interface and the second surface configured to face the tissue site.

7. The dressing of claim 6, wherein the flange extends outward perpendicular to the exterior surface of the sensing conduit, and wherein the flange includes an external diameter that is larger than an external diameter of the sensing conduit.

8. The dressing of claim 6, wherein the mounting surface is configured to be coupled to the cover and a portion of the tissue interface is configured to be captured between the cover and the first surface of the flange of the sensing conduit.

9. The dressing of claim 6, wherein the first surface of the flange is configured to contact the tissue contact surface of the tissue interface.

10. The dressing of claim 9, wherein the second surface of the flange is configured to contact the tissue site.

11. The dressing of claim 6, wherein the flange is coupled to the tissue contact surface of the tissue interface.

12. (canceled)

13. The dressing of claim 3, wherein the second end of the sensing conduit is formed integrally with the tissue contact surface of the tissue interface.

14. The dressing of claim 1, wherein the tissue contact surface of the tissue interface is configured to be positioned in direct contact with the tissue site.

15. (canceled)

16. (canceled)

17. The dressing of claim 6, wherein the tissue interface comprises a film layer having a first side opposite a second side and a plurality of fluid passages disposed through the first side and the second side, the first side of the film layer defining the tissue contact surface of the tissue interface.

18. The dressing of claim 17, wherein the flange of the sensing conduit is positioned on the second side of the film layer with the sensing conduit being in fluid communication with the first side of the film layer through at least one of the plurality of fluid passages.

19. The dressing of claim 17, wherein the flange of the sensing conduit is positioned on the first side of the film layer.

20. The dressing of claim 17, wherein the film layer is a first film layer and wherein the tissue interface further comprises:
a manifold including a first side opposite a second side, the first side of the manifold disposed adjacent to the second side of the first film layer; and
a second film layer including a first side opposite a second side, the first side of the second film layer disposed adjacent to the second side of the manifold and forming at least a portion of the cover.

21. The dressing of claim 20, wherein the flange of the sensing conduit is configured to be positioned between the first film layer and the manifold.

22. (canceled)

23. (canceled)

24. A system including the dressing of claim 1, further comprising:
a negative-pressure source configured to be fluidly coupled to the fluid pathway; and
a pressure sensor configured to be fluidly coupled to the sensing pathway.

25. A dressing interface configured to be coupled to a dressing, the dressing interface comprising:
a housing configured to be coupled to a first portion of the dressing, the housing including a fluid pathway and a sensing pathway;
a sensing conduit configured to pass into a thickness of the dressing, the sensing conduit including a first end configured to be coupled to the sensing pathway and a second end including a flange that is configured to be coupled to a second portion of the dressing, wherein the sensing pathway extends through the sensing conduit to the second portion of the dressing, and wherein the sensing pathway is fluidly isolated from the fluid pathway.

26. (canceled)
